(19) United States
(12) Patent Application Publication
(10) Pub. No.: US 2008/0170988 A1
(43) Pub. Date: Jul. 17, 2008

(54) Method of Preparing Nanoparticles of Alginate-Galactosyl-Polylsine

(76) Inventor: Yangde Zhang, Hunan (CN)

Correspondence Address: KNOBBE MARTENS OLSON & BEAR LLP, 2040 MAIN STREET, FOURTEENTH FLOOR, IRVINE, CA, 92614, US

(21) Appl. No.: 11/663,120

(22) PCT Filed: Jul. 18, 2005
(86) PCT No.: PCT/CN05/01057

§ 371 Date: Mar. 25, 2008

(30) Foreign Application Priority Data
Aug. 19, 2004 (CN) 200410046679.4

(51) Int. Cl. A61K 51/04; C07G 17/00; A61P 35/00

(52) U.S. Cl. 424/1.11; 536/114

(57) ABSTRACT

The technological plan of preparing approach of galactosized algin-polylysine nanoparticle is: synthesize galactosized polylysine by hydrogenization of amine, to apply “finished gelatin of Wharton” to synthesize galactosized algin-polylysine nano gelatin of Wharton: dissolve algin and Calcium chloride in the water respectively, with the magnetic agitation of algin solution, add Calcium chloride solution and galactosized polylysine in turn, standing overnight at ordinary temperature, then get the galactosized algin-polylysine nanoparticle suspl. Nanoparticle suspl. is purified by SephadexG-25. Purified output is dehydrated in vacuum. High liver-target nanogene carrier system from this invention offers theoretical foundation and experimental basis to the targeted gene therapy of primarily hepatoma, and extend to the gene therapy of viral hepatitis and the construction of gene-engineering liver cells.

### BACKGROUND OF THE INVENTION

1. Field of the Invention

The present invention is related to a method for the preparation of nanoparticle medicine of hepatoma gene therapy.

2. Description of the Related Art

Primary hepatic carcinoma is one of the 10 most popular malignant tumors in the world, of which there are some 386 thousand people all over the world die every year, 45% sufferers being Chinese. The conventional therapy has the disadvantages of being low in exairesis rate, being short of Chem targeting and being obvious in toxic and side effects. In recent years, even though the gene therapy has made some progress in basic research, the clinical experiment result is unsatisfying.

### SUMMARY OF THE INVENTION

This invention aims at providing a method for preparation of galactosized algin-polylysine nanoparticle—a gene therapy medicine, which is of good targeting and fewer toxic and side effects.

The technological solution of this invention is to synthesize galactosized polylysine by hydrogenization of amine, and to apply “finished gelatin of Wharton” to synthesize galactosized algin-polylysine nano gelatin of Wharton. The concrete steps are: to dissolve algin and Calcium chloride in the water respectively, with the magnetic agitation of algin solution, add Calcium chloride solution and galactosized polylysine in turn, standing overnight at ordinary temperature, then get the galactosized algin-polylysine nanoparticle suspl. Nanoparticle suspl. is purified by SephadexG-25. Purified output is dehydrated in vacuum.

Algin forms nanoparticle under the complexation of the best polycoagulant Calcium chloride, but the particle diameter is relatively big and tends to become gelatum. Polylysine, as dispersing agent, can make the nanoparticle smaller and better-distributed. If polylysine group PLL is added in first, the nanodiameter is about 850 nm. Hence, it is better to add Calcium chloride first and then polylysine. In this way, the nanoparticle diameter of algin-PLL is about 250 nm, globular, well-distributed and of no aggregation of nanoparticle.

Nanobiotechnology is a scientific field with great potential in 21 century. The appearance of nanotechnology provide a new idea for transferring gene carrier. There are many advantages to transfer nucleotide by nanocarrier: 1. It conduce s cell transfection effectively. Duguid et al have made researches on the relation between the efficiency of gene transfer and the property of nanoparticle. The result shows, the efficiency of gene transfer is influenced by the following factors: content of DNA, porosity of nanoparticle, carrier solution degree of cell resolvase, content of diaminocaproic acid residue. 2. It can transfer diaminocaproic acid to the target. On entering in corpore, the nanoparticles are regulated by many composition of hematoplasma, and are swallowed by mononuclear macrophage. Surface electric charge of nanoparticle medicine Complex, hydrophilia, lipophilia, particle diameter and load of tumor related group decide the adsorption of IgG and C_{3b} in hematoplasma and quantity of encapsulation, and further decide the in vivo metabolism and biodistribution. 3. It can effectively protect nucleotide and prevent it from degrading by biocatalyst. 4. Inorganic nanoparticles have the function of killing cancer cell, and do no harm to normal cells.

High liver-target nanogene carrier system from this invention offers theoretical foundation and experimental basis to the targeted gene therapy of primarily hepatoma, and extend to the gene therapy of viral hepatitis and the construction of gene-engineering liver cells.

### BRIEF DESCRIPTION OF THE DRAWINGS

FIG. 1 is the algin nanoparticle of the invention under electronic microscope. (1:40000)

In the figure, nanoparticle diameter of algin-PLL is about 250 nm, globular, well-distributed and of no aggregation of nanoparticle.

FIG. 2 is the block diagram of algin nanoparticle (AlgNP)-DNA composition.

In the figure, AlgNP, namely algin nanoparticle, is of sponginess, central porosity. It belongs to encapsulative nanoparticle, and encapsulate DNA in the aquosity core.

FIG. 3 is the liver absorption curve of mice after AlgNP and Lac-AlgNP phleb-administration.

FIG. 4 is the diagram of AlgNP and Lac-AlgNP distribution in different organs 30 minutes after administration.

FIG. 5 is the diagram of ¹²⁵I-LacAlgNP(A) and ¹²⁵I-LacAlgNP(B) distribution in mice.

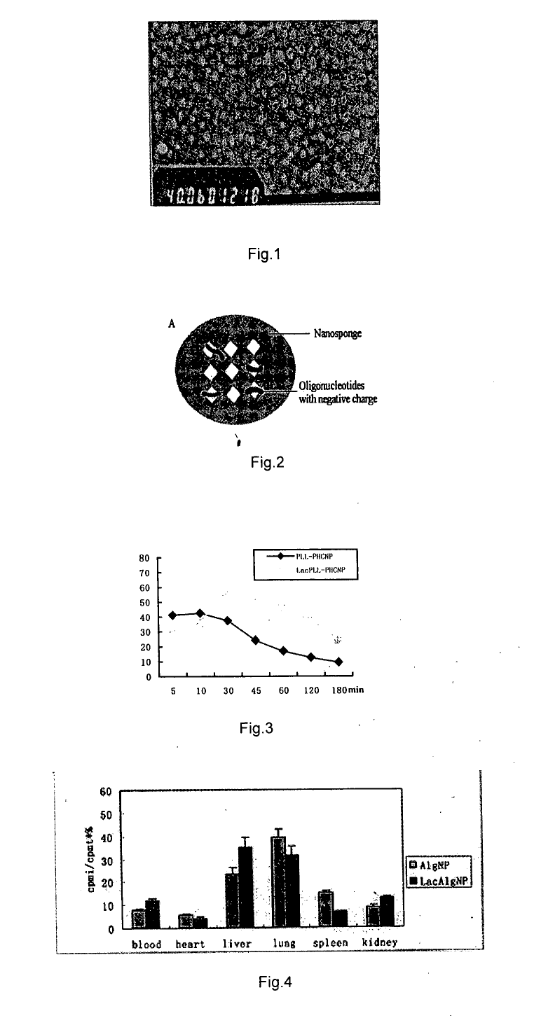

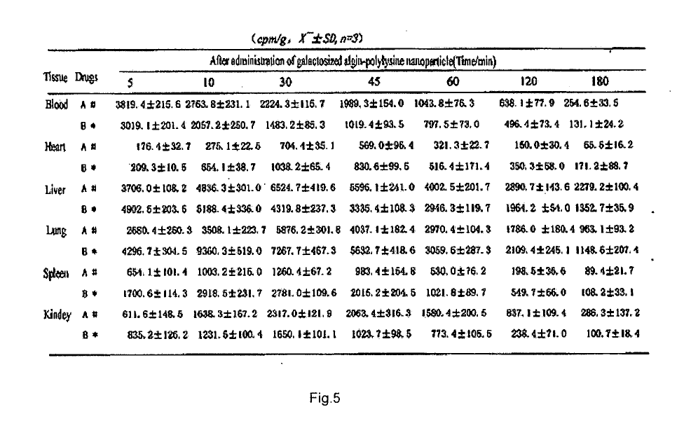

According to FIG. 3, FIG. 4 and FIG. 5, liver absorption curve liver actino-activity of Alg-LacPLLNP is higher than that of Alg-PLLNP, with no significant difference (t 3=1.054, P>0.05). The comparison of Alg-LacPLLNP in different organs shows the significant difference (F=13.282, P<0.05). The diagram of distribution in different mice organs 30 minutes after administration shows Alg-PLLNP targets lung primarily and liver secondarily. Alg-LacPLLNP is increasing obviously in primary liver target comparing with Alg-PLLNP. LSD statistic shows: there is no significant difference between liver and lung, while the difference exists between liver or lung and other organs.

### DETAILED DESCRIPTION OF THE PREFERRED EMBODIMENTS

The following is to introduce the approach of preparing galactosized algin-polylysine nanoparticle with examples.

Compose galactosized polylysine by reduction of amine. Take 200 mg polylysine, alpha-galactose 800 mg, cyan-boronacid sodium 500 mg, dissolve them in 200 ml of ion water, and put it in electric homeothermia box 37 C for 24 h, add adequate NaOH solution into reactive solution with sophisticated acid estimation apparatus, decrease PH value to 8.5, keep it in reaction for 6 h. Reactive solution is dialyzed by water at ordinary temperature, end the reaction and expel inreacted galactosylglucose, purify it by SephadexG-25 column and dehydrate the purified substance in vacuum.

Precisely weigh algin 0.12 g, Calcium chloride 0.2 g, dissolve in 200 ml and 100 ml deionized water respectively. Take 9.5 ml algin solution into 30 ml beaker, agitated with magnet, drop into 0.5 ml Calcium chloride solution, keep agitating for 30 min, add 0.005% galactosized polylysine 2 ml, standing overnight at ordinary temperature, then get the galactosized algin-polylysine nanoparticle suspl. nanoparticle suspl. is purified by Sephadex G-25 and dehydrated the purified substance in vacuum.

1. A method of making a galactosized algin-polylysine nanoparticle comprising:
(a) dissolving algin and Calcium chloride in water;
(b) adding galactosized polylysine to the mixture obtained in step (a); and
(c) isolating said galactosized algin-polylysine nanoparticle from the mixture obtained in step (b).

2. (canceled)

3. The method of claim 1, wherein said galactosized polylysine is prepared by mixing polylysine and alpha-galactose with an acid and incubating said mixture of polylysine, alpha-galactose, and acid at 37° C.

4. The method of claim 3, further comprising adding sufficient NaOH to said mixture of polylysine, alpha-galactose, and acid to adjust the pH of said mixture to 8.5 after incubation at 37° C.

5. The method of claim 3, wherein said mixture is incubated for 24 hours.

6. The method of claim 4, wherein said mixture is maintained at a pH of 8.5 for 6 hours.

7. The method of claim 4, further comprising dialyzing said mixture against water.

8. The method of claim 7, wherein said dialyzed mixture is isolated by chromatography over a SephadexG-25 column.

9. The method of claim 1, further comprising agitating said mixture of algin and Calcium chloride obtained in step (a) with a magnet.

10. The method of claim 1, wherein 0.0005% galactosized polylysine is added to said mixture of algin and Calcium chloride obtained in step (a).

11. The method of claim 1, wherein said galactosized algin-polylysine nanoparticle is isolated by chromatography over a SephadexG-25 column.

12. A method of directing a therapeutic to the liver or lung of a mammal comprising:
providing a galactosized algin-polylysine nanoparticle that comprises a therapeutic to a mammal; and
measuring the presence of the therapeutic in the liver or lung of said mammal.

13. The method of claim 12, wherein said therapeutic is a radioactive material.

14. The method of claim 12, wherein said galactosized algin-polylysine nanoparticle is a galactosized algin-polylysine nanoparticle prepared by the method of claim 1.

15. A galactosized algin-polylysine nanoparticle.
